(19) United States
(12) Patent Application Publication
(10) Pub. No.: US 2017/0001185 A1
(43) Pub. Date: Jan. 5, 2017

(54) MODIFIED PREFORMATION METHOD FOR CATALYST ACTIVATION IN ETHYLENE REACTIONS

(71) Applicant: SAUDI BASIC INDUSTRIES CORPORATION, Riyadh (SA)

(72) Inventors: Roland Schmidt, Wiehl (DE); Mohammed H. Al-Hazmi, Riyadh (SA)

(21) Appl. No.: 15/106,994

(22) PCT Filed: Jan. 5, 2015
(86) PCT No.: PCT/IB2015/050077

§ 371 Date: Jun. 21, 2016

Related U.S. Application Data
(60) Provisional application No. 61/924,064, filed on Jan. 6, 2014.

(51) Int. Cl. B01J 31/14; C07C 2/32; B01J 31/18

(52) U.S. Cl. CPC B01J 31/143; B01J 31/1885; C07C 2/32; B01J 2231/20; B01J 2531/62; C07C 2531/14; C07C 2531/18

(57) ABSTRACT

Systems and methods for catalyst activation in ethylene reactions are described. Systems and methods may include pre-mixing at least one ligand and at least one chromium source in at least one solvent to form a pre-mixed composition; activating the pre-mixed composition with an activator to form an activated composition; and supplying the pre-activated composition to a reactor.

This application is a National Stage application of PCT/IB2015/050077, filed Jan. 5, 2015, which claims the benefit of U.S. Provisional Application No. 61/924,064 filed Jan. 6, 2014, both of which are incorporated by reference in their entirety herein.

### FIELD OF THE INVENTION

The present invention relates to systems and methods for catalyst activation, and more specifically, to integrated systems and methods for catalyst activation in ethylene reactions. Ethylene reactions may include, but are not limited to, oligomerization and polymerization reactions.

### BACKGROUND

Catalyst systems and processes for the oligomerization of ethylene, in particular for the selective trimerization of ethylene to 1-hexene, have been previously described. Existing catalyst compositions typically include a chromium source, a ligand, a solvent and an activator. In existing systems, the ligand and the chromium source are mixed together in a solvent and are activated by an activator prior to use.

Compounds having the general structure PNPNH are known ligand systems that can be successfully used in a catalyst for the oligomerization of ethylene, where they function as ligands to be reacted with a metal, preferably chromium, catalyst. In conjunction with a suitable cocatalyst such catalyst system can be effective in the di-, tri- and/or tetramerization of ethylene.

One known drawback of the prior art catalyst systems used in ethylene oligomerization reactions is the formation of long-chain by-products such as waxes and polyethylene. This is highly undesirable and can lead to fouling of equipment, such as the reactor inner surfaces, heat exchangers, etc. Moreover, wax or polymer formation can lead to plugging of tubing, valves, pumps, and other equipment, resulting in plant down time while purging, cleaning and maintaining affected equipment.

There accordingly remains a need for improved systems and methods for catalyst activation in ethylene oligomerization and polymerization reactions to improve catalyst performance.

### SUMMARY

Embodiments of the present invention solve many of the problems and/or overcome many of the drawbacks and disadvantages of the prior art by providing systems and methods for catalyst activation in ethylene reactions. Ethylene reactions include, but are not limited to, oligomerization and polymerization reactions.

Embodiments of the present invention include systems and methods for catalyst activation in ethylene reactions. The systems and methods include pre-mixing at least one ligand and at least one chromium source in at least one solvent to form a pre-mixed composition; activating the pre-mixed composition with an activator to form an activated composition; and supplying the pre-activated composition to a reactor.

Additional features, advantages, and embodiments of the invention are set forth or apparent from consideration of the following detailed description, drawings, and claims. Moreover, it is to be understood that both the foregoing summary of the invention and the following detailed description are exemplary and intended to provide further explanation without limiting the scope of the invention as claimed.

### BRIEF DESCRIPTION OF THE DRAWINGS

The accompanying drawings, which are included to provide a further understanding of the invention and are incorporated in and constitute a part of this specification, illustrate preferred embodiments of the invention and together with the detailed description serve to explain the principles of the invention. In the drawings:

FIG. 1 shows an exemplary system for pre-activating a catalyst according to an embodiment.

FIG. 2 shows a graph of ethylene uptake over time based on stirring times as per Example 1, according to an embodiment.

FIG. 3 shows a graph of reaction temperature over time as per Example 1, according to an embodiment.

FIG. 4 shows a graph of ethylene uptake over time based on a modified system as per Example 2, according to an embodiment.

### DETAILED DESCRIPTION

Systems and methods are described for integrated processes for catalyst activation in ethylene reactions. Ethylene reactions may include, but are not limited to, oligomerization reactions and polymerization reactions. Specific reactions may include trimerization reactions, dimerization reactions, tetramerization reactions, Schulz-Flory distribution oligomerizations, and others.

The processes described herein are exemplary processes only and used for illustrative purposes. Other variations and combinations of steps and components may be used as necessary.

Certain embodiments described herein may be directed to a selective ethylene reaction, such as a 1-hexene ethylene trimerization process, using a preformation composition. The preformation composition may include various components. In certain embodiments, the preformation composition may include (1) a ligand, (2) a chromium source, (3) a solvent, and (4) an activator. A catalyst modifier is preferably present. It should be understood that each of these components of the preformation composition may have one or more constituents. For example, the chromium source may be multiple sources of chromium used together to supply the desired amount of chromium.

The ligand may be one or more compounds. In certain embodiments, the ligand may be ((phenyl)₂PN(isopropyl)P(phenyl)NH(isopropyl)) (PHPNH). In certain embodiments, the ligand may have a general structure R₁R₂P—N(R₃)—P(R₄)—N(R₅)—H, wherein R₁, R₂, R₃, R₄ and R₅ are independently selected from hydrogen, halogen, (substituted) amino, trialkylsilyl, (substituted) phosphino, C₁-C₁₅-alkyl and/or alkenyl and/or alkynyl, aryl and substituted aryl. In certain embodiments, the ligand may be Ph₂PN(iPr)P(Ph)N(iPr)H.

In particular, the ligand is a PNPNH compound, which as used herein has the general structure R₁R₂P—N(R₃)—P(R₄)—N(R₅)—H, wherein R₁, R₂, R₃, R₄ and R₅ are independently hydrogen, halogen, substituted or unsubstituted amino, substituted or unsubstituted tri(C₁₋₆-alkyl)silyl, preferably trimethylsilyl, substituted or unsubstituted phosphino, substituted or unsubstituted C₁-C₁₀-alkyl, or substituted or unsubstituted C₆-C₂₀-aryl, or any cyclic derivative wherein at least one of the P or N atoms is a member of a ring system, the ring system being formed from one or more constituent compounds of the PNPNH compound by substitution, i.e. by formally eliminating per constituent compound either two whole groups R₁-R₅ (as defined) or H, one atom from each of two groups R₁-R₅ (as defined) or a whole group R₁-R₅ (as defined) or H and an atom from another group R₁-R₅ (as defined), and joining the formally so created valence-unsaturated sites by one covalent bond per constituent compound to provide the same valence as initially present at a given site. A combination of different ligands can be used. Suitable cyclic derivatives can be as follows:

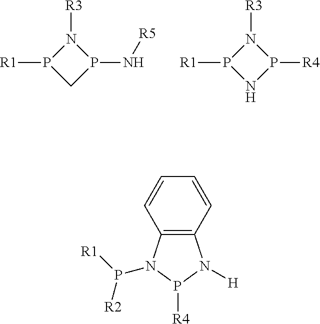

In a specific embodiment, R₁, R₂, R₃, R₄ and R₅ are independently hydrogen, substituted or unsubstituted C₁-C₈-alkyl, or substituted or unsubstituted C₆-C₂₀-aryl, more preferably unsubstituted C₁-C₆-alkyl or unsubstituted C₆-C₁₀-aryl.

In certain embodiments, the chromium compound may be include organic or inorganic salts, coordination complexes, and organometallic complexes of Cr(II) or Cr(III). Preferably the chromium compound is CrCl₃(THF)₃, Cr(III)acetylacetonate, Cr(III)octanoate, chromium hexacarbonyl, Cr(III)-2-ethylhexanoate, benzene(tricarbonyl)-chromium or Cr(III)chloride. A combination of different chromium compounds can be used.

In certain embodiments, examples of the solvent include one or more of an aromatic or aliphatic solvent or combinations thereof, preferably toluene, benzene, ethylbenzene, cumenene, xylenes, mesitylene, C₄-C₁₅ paraffins, cyclohexane, C₄-C₁₂ olefins, such as butene, hexene, heptene, octene, or ethers or multiethers, such as diethylether, tetrahydrofuran, dioxane, di(C₁-C₈-alkyl)ethers, more preferably an aromatic solvent, most preferably toluene.

In certain embodiments, the activator may be triethylaluminum. In certain embodiments, the activator may be one or more of a tri(C₁-C₆-)alkyl aluminum, C₁-C₆-alkyl aluminum sesquichloride, di(C₁-C₆-)alkyl aluminum chloride, C₁-C₆-alkyl aluminum dichloride, wherein alkyl is preferably methyl, ethyl, isopropyl, or isobutyl, a methylaluminoxane (MAO) or combinations thereof.

A modifier can also be present in the catalyst composition, for example an ammonium or phosphonium salt of the type [H₄E]X, [H₃ER]X, [H₂ER₂]X, [HER₃]X, or [ER₄]X wherein E is N or P, X is Cl, Br or I, and each R is independently substituted or unsubstituted C₁-C₂₂-alkyl, substituted or unsubstituted C₃-C₁₀-cycloalkyl, substituted or unsubstituted C₂-C₂₂-acyl, substituted or unsubstituted C₆-C₃₀-aryl, substituted or unsubstituted C₂-C₂₂-alkenyl, substituted or unsubstituted C₂-C₂₂-alkynyl or the corresponding bridging di-, tri- or multiunits, or ammonium or phosphonium salts based on cyclic amines or cyclic phosphines. In some embodiments each R is independently substituted or unsubstituted C₁-C₁₈-alkyl, substituted or unsubstituted C₃-C₆-cycloalkyl, substituted or unsubstituted C₂-C₁₈-acyl, substituted or unsubstituted C₆-C₁₈-aryl, substituted or unsubstituted C₂-C₁₈-alkenyl, substituted or unsubstituted C₂-C₂₂-alkynyl; or more preferably C₁-C₁₄-alkyl, C₂-C₁₄-acyl, or phenyl or naphthyl. Preferably, the modifier is dodecyltrimethylammonium chloride or tetraphenylphosphonium chloride. The modifier can modify the activator, and serve as a chlorine source.

A pre-activation step is used to improve catalyst performance. The pre-activation step may be combined with the use of a higher concentrated solution, i.e., using less solvent, to further improve catalyst performance. Concentration (catalyst/solvent) may be from approximately 0.001% to approximately 10%, more preferably from approximately 0.001% to approximately 5%, and more preferably from 0.001% to approximately 1%.

In certain embodiments, a ligand and a chromium source are mixed together in a solvent in a pre-activation step and then activated by an activator prior to use. In certain exemplary embodiments, a ligand such as PNPNH, and a chromium source, such as chromium chloride and chromium acetyl acetonate, may be mixed together in a solvent, such as toluene, and activated by an activator, such as triethylaluminum, prior to use. If used, the catalyst modifier can be added with the ligand and/or the chromium source, or with the activator.

In certain embodiments, (1) a pre-activation step, and (2) a modified concentration of the solution, i.e., less toluene, may improve catalyst performance significantly. The catalyst activity may be more than doubled when all components were mixed externally and stirred prior to transfer to the reactor.

Excessive pre-activation time, however, may decrease activity again. In certain embodiments the pre-activation time should not exceed approximately 3 to approximately 5 hours. In certain embodiments, the overall activity may decrease with prolonged activation time allotment.

In certain embodiments, the ligand and chromium source (and optional modifier) are mixed together in the solvent. Once the components are mixed, they may be continuously or intermittently stirred. Preferably, the mixed components are continuously stirred. The components may be added in sequence to a mixing device at ambient or other conditions.

Mixing may take place for between approximately 1 minute and approximately 18 hours, more preferably, between approximately 10 minutes and approximately 8 hours, and more preferably between approximately 15 minutes and approximately 5 hours.

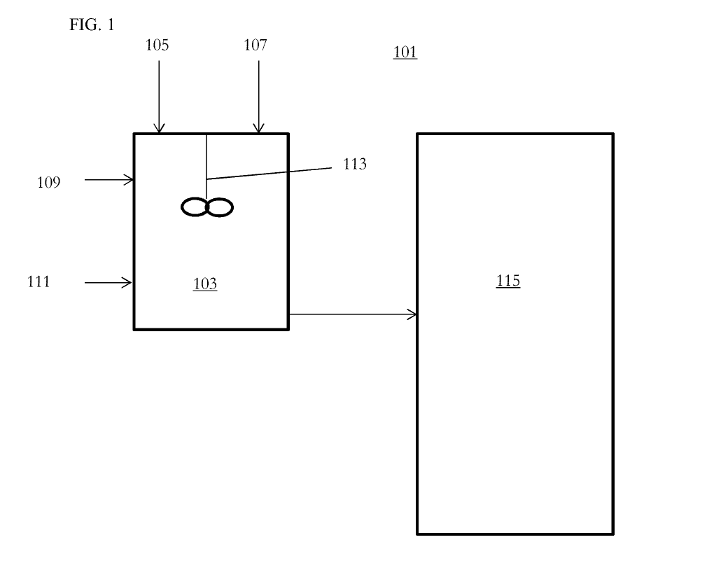

As shown in FIG. 1, a system 101 may provide for pre-activation of a preformation composition. In certain embodiments, a preformation unit 103 may prepare a preformation composition for the oligomerization of ethylene. The preformation unit 103 may receive ligand 105, chromium 107 and solvent 109. The preformation unit 103 may then receive an activator 111. The preformation unit 103 may include a stirrer 113 for mixing the preformation composition prior to delivering the preformation composition to a reactor 115. Each line into the preformation unit may, optionally, each having dosing pumps and/or valves. Preferably, inert conditions may be used. In a preferred embodiment, the system is integrated with an apparatus for the oligomerization of ethylene, more preferably for an apparatus for the trimerization of ethylene to 1-hexene, wherein reactor 115 is suitable for the oligomerization or the trimerization and is fitted with an outlet for the oligomeric product or the 1-hexene (not shown). Other components of such apparatuses are known in the art.

The following Examples are provided are for illustrative purposes only and are not to be construed as limiting in any manner.

### Example 1

### Ethylene Trimerization

A 300 ml pressure reactor was equipped with a dip tube, thermowell, gas entrainment stirrer, cooling coil, control units for temperature, pressure, and stirrer speed. The components of the pressure reactor were each connected to a data acquisition system. The pressure reactor was inertized with dry nitrogen and filled with 100 ml anhydrous toluene. 68 mg of the ligand ((phenyl)₂PN(isopropyl)P(phenyl)NH(isopropyl)) in 1.5 ml toluene was combined with 37 mg CrCl₃(THF)₃ (THF=tetrahydrofuran) under a nitrogen blanket. This catalyst solution was stirred for various times prior to being transferred to the reactor under constant nitrogen flow, along with 1.7 ml of a 1.9 mol/l solution of triethylaluminum (TEA) in toluene.

The reactor was sealed, pressurized with 30 bar dry ethylene, and heated to 40° C. While stirring at 1200 rpm, the ethylene consumption was monitored by the data acquisition system and an electronic balance by constantly weighing the ethylene pressure cylinder. After 120 min residence time, the reaction in the liquid phase was quenched by transferring the liquid inventory by means of ethylene pressure to a glass vessel filled with approximately 100 ml water. The entire gas phase from the reactor's head space was quantified by calibrated gas meter and was then collected quantitatively in a purged and evacuated gas bag.

After separation of the liquid organic phase, the total mass was determined by weighing. Subsequently, the composition of the organic phase was analyzed by gas chromatography/flame ionization detection (GC/FID). The previously collected gas phase was analyzed separately by GC/FID.

Based on the measured data, the mass balance was closed and the overall yields and selectivities were determined. See Table 1 below for activity information.

TABLE 1

| Time (h) | Activity (kg/g Cr · h) |
|---|---|
| Standard | 9 |
| 1 | 15.3 |
| 3 | 19.2 |
| 18 | 14.4 |
| 25* | 3.65 |

*Continuous stirring

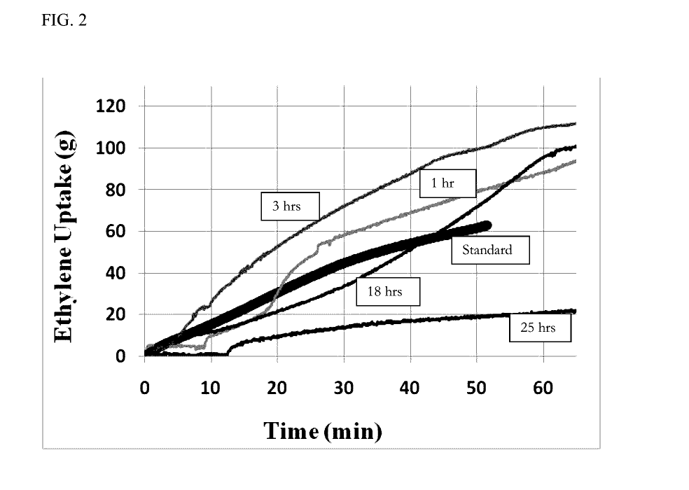

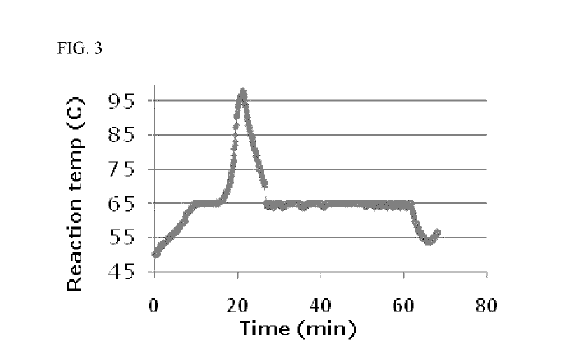

See FIG. 2 for ethylene uptake over time. See FIG. 3 for reaction temperature over time.

### Example 2

### Modified Ethylene Trimerization

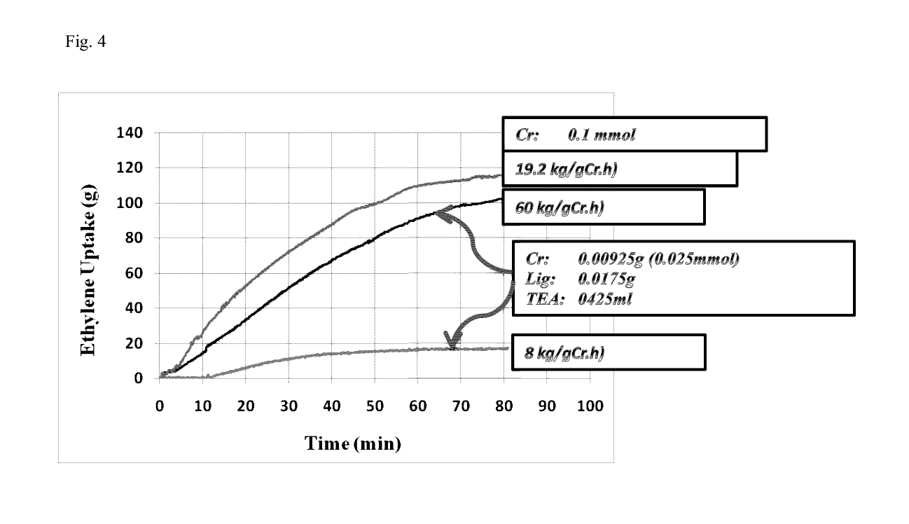

In FIG. 4 is shown a standard run (60 kg product) and two curves with an unoptimized, longer (bottom curve) and an optimized, shorter (middle curve) pre-activation time for the chromium compound and the ligand, illustrating that the unoptimized, longer activation time leads to reduced activity at the same concentration of chromium and the other catalyst components. The top and the bottom lines had the same activation time, but an increased concentration of chromium (0.1 mmol for the top line, 0.025 for the middle line), which indicates that the improved production is not a concentration effect but primarily a pre-activation effect.

In addition, general observations (data not shown) include that the modified process advantageously resulted in very low polymer formation as evidenced by a very clear polymer solution. In a further advantage there was also better reaction temperature control.

The invention is further illustrated by the following embodiments.

### Embodiment 1

A method for improving catalyst performance, preferably for improving catalyst performance in an oligomerization of ethylene, more preferably for improving catalyst performance in a trimerization of ethylene to 1-hexene, the method comprising pre-mixing at least one ligand and at least one chromium source in at least one solvent to form a pre-mixed composition; activating the pre-mixed composition with an activator to form an activated composition; and supplying the pre-activated composition to a reactor.

### Embodiment 2

The method of embodiment 1, wherein the ligand is ((phenyl)₂ PN(isopropyl) P(phenyl)NH(isopropyl)) (PHPNH).

### Embodiment 3

The method of any one or more of embodiments 1 to 2, wherein the chromium source is selected from the group consisting of: chromium chloride, chromium acetyl acetonate, and combinations thereof.

### Embodiment 4

The method of any one or more of embodiments 1 to 3, wherein the solvent is toluene.

### Embodiment 5

The method of any one or more of claims 1 to 4, wherein the solvent is supplied at a concentration between approximately 0.1% and approximately 95%.

### Embodiment 6

The method of any one or more of claims 1 to 5, wherein the activator is triethylaluminum.

### Embodiment 7

The method of any one or more of embodiments 1 to 6, wherein the activating comprises mixing external to the reactor and stirring.

### Embodiment 8

The method of embodiment 7, wherein the mixing time is between approximately 1 minute and approximately 18 hours.

### Embodiment 9

A method for improving catalyst performance in an oligomerization of ethylene, more preferably for improving catalyst performance in a trimerization of ethylene to 1-hexene, the method comprising: pre-mixing ((phenyl)₂ PN(isopropyl) P(phenyl)NH(isopropyl)) and at least one chromium source in toluene to form a pre-mixed composition; activating the pre-mixed composition with an activator to form an activated composition; and supplying the pre-activated composition to a reactor.

### Embodiment 10

The method of embodiment 9, wherein the chromium source is selected from the group consisting of: chromium chloride, chromium acetyl acetonate, and combinations thereof.

### Embodiment 11

The method of embodiment 9 or 10, wherein the toluene is supplied at a concentration between approximately 0.1% and approximately 95%.

### Embodiment 12

The method of any one or more of embodiments 9 to 11, wherein the activator is triethylaluminum.

### Embodiment 13

The method of any one or more of embodiments 9 to 12, wherein the activating comprises mixing external to the reactor and stirring.

### Embodiment 14

The method of embodiment 13, wherein the mixing time is between approximately 1 minute and approximately 18 hours.

### Embodiment 15

A system for improving catalyst performance, preferably for improving catalyst performance in an oligomerization of ethylene, more preferably for improving catalyst performance in a trimerization of ethylene to 1-hexene, the system comprising: a pre-mixing chamber for receiving inputs of one or more ligands, one or more chromium sources, one or more solvents, and one or more activators; one or more stirrers; and a reaction vessel in fluid communication with the pre-mixing chamber for receiving a pre-activated preformation composition.

### Embodiment 16

The system of embodiment 15, wherein the one or more ligands and one or more chromium sources are supplied simultaneously.

### Embodiment 17

The system of embodiment 15 or 16, wherein the ligand is ((phenyl)₂PN(isopropyl)P(phenyl)NH(isopropyl)) (PHPNH).

### Embodiment 18

The system of any one or more of embodiments 15 to 17, wherein the chromium source is selected from the group consisting of: chromium chloride, chromium acetyl acetonate, and combinations thereof.

### Embodiment 19

The system of any one or more of embodiments 15 to 18, wherein the solvent is toluene.

### Embodiment 20

The system of any one or more of embodiments 15 to 18, wherein the activator is triethylaluminum.

### Embodiment 21

The systems and methods described herein.

In general, the invention can alternatively comprise, consist of, or consist essentially of, any appropriate components herein disclosed. The invention can additionally, or alternatively, be formulated so as to be devoid, or substantially free, of any components, materials, ingredients, adjuvants or species used in the prior art compositions or that are otherwise not necessary to the achievement of the function and/or objectives of the present invention.

The singular forms “a,” “an,” and “the” include plural referents unless the context clearly dictates otherwise. “Or” means “and/or.” The endpoints of all ranges directed to the same component or property are inclusive and independently combinable. Disclosure of a narrower range or more specific group in addition to a broader range is not a disclaimer of the broader range or larger group. Unless defined otherwise, technical and scientific terms used herein have the same meaning as is commonly understood by one of skill in the art to which this invention belongs. A “combination” is inclusive of blends, mixtures, alloys, reaction products, and the like. All cited patents, patent applications, and other references are incorporated herein by reference in their entirety. However, if a term in the present application contradicts or conflicts with a term in the incorporated reference, the term from the present application takes precedence over the conflicting term from the incorporated reference.

As used herein, the term “alkyl” means a branched or straight chain, saturated, monovalent hydrocarbon group, e.g., methyl, ethyl, i-propyl, and n-butyl. “Alkylene” means a straight or branched chain, saturated, divalent hydrocarbon group (e.g., methylene (—CH₂—) or propylene (—(CH₂)₃—)). “Alkynyl” means a straight or branched chain, monovalent hydrocarbon group having at least one carbon-carbon triple bond (e.g., ethynyl). “Alkoxy” means an alkyl group linked via an oxygen (i.e., alkyl-O—), for example methoxy, ethoxy, and sec-butyloxy. “Cycloalkyl” means a monovalent cyclic hydrocarbon group of the formula —CₙH₂ₙ₋ₓ wherein x is the number of cyclization(s). “Aryl” means a monovalent, monocyclic or polycyclic, aromatic group (e.g., phenyl or naphthyl). The prefix “halo” means a group or compound including one more halogen (F, Cl, Br, or I) substituents, which can be the same or different. The prefix “hetero” means a group or compound that includes at least one ring member that is a heteroatom (e.g., 1, 2, or 3 heteroatoms, wherein each heteroatom is independently N, O, S, or P.

“Substituted” means that the compound or group is substituted with at least one (e.g., 1, 2, 3, or 4) substituents instead of hydrogen, where each substituent is independently nitro (—NO₂), cyano (—CN), hydroxy (—OH), halogen, thiol (—SH), thiocyano (—SCN), C₁₋₆ alkyl, C₂₋₆ alkenyl, C₂₋₆ alkynyl, C₁₋₆haloalkyl, C₁₋₉ alkoxy, C₁₋₆ haloalkoxy, C₃₋₁₂ cycloalkyl, C₅₋₁₈ cycloalkenyl, C₆₋₁₂ aryl, C₇₋₁₃ arylalkylene (e.g, benzyl), C₇₋₁₂ alkylarylene (e.g, toluyl), C₄₋₁₂ heterocycloalkyl, C₃₋₁₂ heteroaryl, C₁₋₆ alkyl sulfonyl (—S(═O)₂-alkyl), C₆₋₁₂ arylsulfonyl (—S(═O)₂-aryl), or tosyl (CH₃C₆H₄SO₂—), provided that the substituted atom's normal valence is not exceeded, and that the substitution does not significantly adversely affect the manufacture, stability, or desired property of the compound. When a compound is substituted, the indicated number of carbon atoms is the total number of carbon atoms in the group, including those of the substituent(s).

While the invention has been described with reference to an exemplary embodiment or embodiments, it will be understood by those skilled in the art that various changes can be made and equivalents can be substituted for elements thereof without departing from the scope of the invention. In addition, many modifications can be made to adapt a particular situation or material to the teachings of the invention without departing from the essential scope thereof.

What is claimed is:

1. A method for improving catalyst performance in an oligomerization of ethylene, the method comprising:
pre-mixing at least one ligand and at least one chromium source in at least one solvent to form a pre-mixed composition;
activating the pre-mixed composition with an activator to form an activated composition; and
supplying the pre-activated composition to a reactor.

2. The method of claim 1, wherein the ligand is ((phenyl)₂PN(isopropyl)P(phenyl)NH(isopropyl)).

3. The method of claim 1, wherein the chromium source is chromium chloride, chromium acetyl acetonate, or a combination comprising at least one of the foregoing.

4. The method of claim 1, wherein the solvent comprises toluene.

5. The method of claim 1, wherein the solvent is supplied at a concentration between approximately 0.1% and approximately 95%.

6. The method of claim 1, wherein the activator is triethylaluminum.

7. The method of claim 1, wherein the activating comprises mixing the pre-mixed composition with the activator external to the reactor and stirring.

8. The method of claim 7, wherein the mixing time is between approximately 1 minute and approximately 18 hours.

9. A method for improving catalyst performance in an oligomerization of ethylene, the method comprising:
pre-mixing ((phenyl)₂PN(isopropyl)P(phenyl)NH(isopropyl)) and at least one chromium source in toluene to form a pre-mixed composition;
activating the pre-mixed composition with an activator to form an activated composition; and
supplying the pre-activated composition to a reactor.

10. The method of claim 9, wherein the chromium source is chromium chloride, chromium acetyl acetonate, or a combination comprising at least one of the foregoing.

11. The method of claim 9, wherein the toluene is supplied at a concentration between approximately 0.1% and approximately 95%.

12. The method of claim 9, wherein the activator is triethylaluminum.

13. The method of claim 9, wherein the activating comprises mixing external to the reactor and stirring.

14. The method of claim 13, wherein the mixing time is between approximately 1 minute and approximately 18 hours.

15. A system for improving catalyst performance in an oligomerization of ethylene, the system comprising:
a pre-mixing chamber for receiving inputs of one or more ligands, one or more chromium sources, one or more solvents, and one or more activators;
one or more stirrers; and
a reaction vessel in fluid communication with the pre-mixing chamber for receiving a pre-activated preformation composition.

16. The system of claim 15, wherein the one or more ligands and one or more chromium sources are supplied simultaneously.

17. The system of claim 15, wherein the ligand is ((phenyl)₂PN(isopropyl)P(phenyl)NH(isopropyl)).

18. The system of claim 15, wherein the chromium source is chromium chloride, chromium acetyl acetonate, and combinations thereof.

19. The system of claim 15, wherein the solvent is toluene.

20. The system of claim 15, wherein the activator is triethylaluminum.
